(19) United States
(12) Patent Application Publication
(10) Pub. No.: US 2020/0279137 A1
(43) Pub. Date: Sep. 3, 2020

(54) Systems and Methods for Evaluating Artificial Intelligence Applications in Clinical Practice

(71) Applicant: The Board of Trustees of the Leland Stanford Junior University, Stanford, CA (US)

(72) Inventor: Daniel L. Rubin, Stanford, CA (US)

(73) Assignee: The Board of Trustees of the Leland Stanford Junior University, Stanford, CA (US)

(21) Appl. No.: 16/805,124

(22) Filed: Feb. 28, 2020

Related U.S. Application Data
(60) Provisional application No. 62/812,905, filed on Mar. 1, 2019.

(51) Int. Cl. G06K 9/62; G06F 16/2457; G06F 16/53; G06F 16/51; G06N 5/04; G16H 30/20; G16H 30/40; G16H 50/20

(52) U.S. Cl. CPC G06K 9/6262; G06F 16/24578; G06F 16/53; G06F 16/51; G06N 5/043; G06K 2209/05; G16H 30/40; G16H 50/20; G06K 9/6267; G06K 9/6254; G16H 30/20

(57) ABSTRACT

Systems and methods for evaluating artificial intelligence applications with seamlessly embedded features in accordance with embodiments of the invention are illustrated. One embodiment includes an AI evaluation system including a plurality of collection servers, an AI evaluation server connected to the plurality of collection servers, including at least one processor and a memory, containing an AI evaluation application that directs the processor to obtain a plurality of ground truth data from the plurality of collection servers, where the ground truth data includes a plurality of image and annotation pairs, generate a first plurality of outputs by providing a first AI system with images from the plurality of image and annotation pairs, compare the first plurality of outputs with annotations from the plurality of image and annotation pairs, generate a first ranking metric of the first AI system based on the comparison, and store the first ranking metric in a database.

### CROSS-REFERENCE TO RELATED APPLICATIONS

The current application claims the benefit of and priority under 35 U.S.C. § 119(e) to U.S. Provisional Patent Application No. 62/812,905 entitled “Evaluating Artificial Intelligence Applications in Clinical Practice” filed Mar. 1, 2019. The disclosure of U.S. Provisional Patent Application No. 62/812,905 is hereby incorporated by reference in its entirety for all purposes.

### STATEMENT OF FEDERALLY SPONSORED RESEARCH

This invention was made with Government support under contracts CA142555 and CA190214 awarded by the National Cancer Institute. The Government has certain rights in the invention.

### FIELD OF THE INVENTION

The present invention generally relates to the performance evaluation of AI systems, and specifically, ensuring that AI systems provide accurate, reliable information in a clinical setting.

### BACKGROUND

Artificial Intelligence (AI) is a field of computer science concerned with creating systems which mimic human actions. A subfield of AI with has yielded fruitful results is machine learning, which is concerned with programs which automatically learn and improve through operation.

### SUMMARY OF THE INVENTION

Systems and methods for evaluating artificial intelligence applications with seamlessly embedded features in accordance with embodiments of the invention are illustrated. One embodiment includes an AI evaluation system including a plurality of collection servers, an AI evaluation server connected to the plurality of collection servers, including at least one processor and a memory, containing an AI evaluation application that directs the processor to obtain a plurality of ground truth data from the plurality of collection servers, where the ground truth data includes a plurality of image and annotation pairs, generate a first plurality of outputs by providing a first AI system with images from the plurality of image and annotation pairs, compare the first plurality of outputs with annotations from the plurality of image and annotation pairs, generate a first ranking metric of the first AI system based on the comparison, and store the first ranking metric in a database.

In another embodiment, the AI evaluation application further directs the processor to generate a second plurality of outputs by providing a second AI system with images from the plurality of image and annotation pairs, compare the second plurality of outputs with annotations from the plurality of image and annotation pairs, generate a second ranking metric of the second AI system based on the comparison, store the second ranking metric in the database, and recommend an AI system for a particular purpose based on the ranking metrics in the database in response to a query.

In a further embodiment, wherein images in the plurality of image and annotation pairs are radiology images.

In still another embodiment, the ground truth data conforms to the Annotation and Image Markup (AIM) file standard.

In a still further embodiment, collection servers in the plurality of collection servers are hospital servers.

In yet another embodiment, the ground truth data is deidentified.

In a yet further embodiment, an annotation of an image and annotation pair identifies whether a disease indicator is present in an image in the image and annotation pair.

In another additional embodiment, an annotation of an image and annotation pair is the output of the first AI system and an agree/disagree indicator by a radiologist of the output of the first AI system.

In a further additional embodiment, the ground truth data is divided into different classifications by image type.

In another embodiment again, the system further includes an input device connected to at least one collection server in the plurality of collection servers, where the input device is running the ePAD application.

In a further embodiment again, a method of evaluating AI includes obtaining a plurality of ground truth data from a plurality of collection servers, where the ground truth data includes a plurality of image and annotation pairs, using an AI evaluation server, generating a first plurality of outputs by providing a first AI system with images from the plurality of image and annotation pairs, using the AI evaluation server, comparing the first plurality of outputs with annotations from the plurality of image and annotation pairs, using the AI evaluation server, generating a first ranking metric of the first AI system based on the comparison, using the AI evaluation server, and storing the first ranking metric in a database, using the AI evaluation server.

In still yet another embodiment, the method further includes generating a second plurality of outputs by providing a second AI system with images from the plurality of image and annotation pairs, using the AI evaluation server, comparing the second plurality of outputs with annotations from the plurality of image and annotation pairs, using the AI evaluation server, generating a second ranking metric of the second AI system based on the comparison, using the AI evaluation server, storing the second ranking metric in the database, using the AI evaluation server, and recommending an AI system for a particular purpose based on the ranking metrics in the database in response to a query, using the AI evaluation server.

In a still yet further embodiment, images in the plurality of image and annotation pairs are radiology images.

In still another additional embodiment, the ground truth data conforms to the Annotation and Image Markup (AIM) file standard.

In a still further additional embodiment, collection servers in the plurality of collection servers are hospital servers.

In still another embodiment again, the ground truth data is deidentified.

In a still further embodiment again, an annotation of an image and annotation pair identifies whether a disease indicator is present in an image in the image and annotation pair.

In yet another additional embodiment, an annotation of an image and annotation pair is the output of the first AI system and an agree/disagree indicator by a radiologist of the output of the first AI system.

In a yet further additional embodiment, the ground truth data is divided into different classifications by image type.

In yet another embodiment again, the method further comprises receiving ground truth data using an input device connected to at least one collection server in the plurality of collection servers, where the input device is running the ePAD application.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures, which are presented as exemplary embodiments of the invention and should not be construed as a complete recitation of the scope of the invention.

FIG. 1 illustrates an AI evaluation system in accordance with an embodiment of the invention.

FIG. 2 illustrates an AI evaluation server in accordance with an embodiment of the invention.

FIG. 3 is a flowchart for an AI evaluation process in accordance with an embodiment of the invention.

### DETAILED DESCRIPTION

Artificial intelligence (AI) technologies are developing rapidly, and there is an explosion in commercial activity in developing AI applications. However, AI tend to be “black boxes” in their operation, and in many fields, this is a cause for concern. For example, in the medical space, where AI systems are relied upon for diagnostics and treatment, it is critical to ensure that the system is producing the correct outcome. Because it can be difficult to tease apart the actual operation of a learned system, systems and methods described herein provide mechanisms for evaluating and validating AI system performance.

With specific respect to the field of radiology, AI systems can be useful in processing medical images and searching for diagnostic markers. Consequently, AI products have the potential of improving radiology practice, but clinical radiology practices lack resources and processes for evaluating whether these products perform as well as advertised in their patient populations. AI algorithms that perform well on data that the vendor acquired during development of those algorithms may not perform as well at institutions who deploy these tools in their patient population. This is referred to “generalizability” of the AI algorithm and it has been shown several times that generalizability in performance of AI algorithms fails at new institutions, requiring a separate evaluation at each institution before the AI algorithm can be deployed there.

Further, even if AI algorithms perform well initially in a particular clinical practice, imaging methods and patient populations in that practice may change over time, and the performance of AI algorithms may thus change over time. Thus, ongoing monitoring of performance of these tools is important. However, at present, clinical practices lack the means to evaluate how well commercial AI tools work in their local patient populations. Conventional best practices include deploying the vendor tool and qualitatively evaluate how well the tool works with their local data. Once deployed, there is little to no ability to monitor ongoing performance of the AI algorithms. Indeed, the U.S. Food and Drug Administration (FDA) has recently proposed a regulatory framework for AI that that specifies a need for post-marketing surveillance, but idiosyncrasies across hospitals such as, but not limited to, different terminologies, different formats, and edits made to local AI system outputs by radiologists have hampered development.

In contrast, in various embodiments, clinical practices can use disclosed systems and methods to evaluate the performance of AI systems based on the practice's local institutional data despite each institution having different practices. Systems and methods described herein can helps the practice to acquire and create a ground truth dataset for testing the AI produce, and permit them to define and measure clinically-relevant metrics for AI performance using those data. In numerous embodiments, patient data from clinical practices is used to establish a registry of AI algorithm performance. Systems for acquiring data and validating AI systems are discussed below.

### AI Evaluation Systems

AI evaluation systems are capable of aggregating ground truth data from multiple independent institutions and evaluating AI systems that are in use, or prospectively useful to said institutions. In numerous embodiments, AI evaluation systems maintain a database of evaluated AI systems according to one or more metrics dependent upon their clinical use. AI evaluation systems can be architected in any number of ways, including as a distributed system. An AI evaluation system in accordance with an embodiment of the invention is described below.

AI evaluation system 100 includes collection servers 110. Collection servers acquire and store ground truth data from local clinics. Collection servers are connected to input devices 112. The input devices can enable trained professionals to input and label data for storage on collection servers. In numerous embodiments, Collection servers and input devices are implemented using the same hardware. In many embodiments, input devices provide access to collection server applications. In various embodiments, input devices are personal computers, cell phones, tablet computers, and/or any other input device as appropriate to the requirements of specific applications of embodiments of the invention. In many embodiments, medical imaging devices can directly upload image data to input devices and/or collection servers.

In many embodiments, the collection servers store a tool for generating and maintaining a database of images, text reports, and/or clinical data that is populated by a collection of cases that the respective institution identifies for evaluating AI systems. These collected data can be used as part of the ground truth data set. For example, in various embodiments, data for the ground truth data set can be identified by a radiology practice searching its reports for cases that are relevant to the AI product under consideration, e.g., cases of chest CT in which lung nodules were identified. In many embodiments, collection servers and/or input devices includes the ePAD application published by Stanford University that receives images that are transmitted to it via the Digital Imaging and Communications in Medicine (DICOM) send protocol from the hospital picture archiving and communication system (PACS).

Further, in numerous embodiments, the collections server and/or input device includes a component that deidentifies the images prior to being received by ePAD (for example, using the Clinical Trial Processor system) if such deidentification is desired. The ePAD application can also receive text reports and other clinical data that establish labels for the images (e.g., treatments, patient survival). Associating clinical data and other key metadata needed for evaluating AI performance in test cases (e.g., the radiologist reading the case, the institution, and imaging equipment/parameters) can be collected and stored as metadata. In various embodiments, the Annotation and Image Markup (AIM) standard is used ‘6-8” for recording this information and making the linkage for each case.

Data from the collection servers are transmitted over a network 120 to a central AI evaluation server 130. The network can be any network capable of transmitting data, including, but not limited to, the Internet, a wired network, a wireless network, and/or any other network as appropriate to the requirements of specific applications of embodiments of the invention. AI evaluation servers, like collection servers, can be implemented as a single server, or as a cluster of connected devices. AI evaluation servers are discussed in further detail below.

### AI Evaluation Servers

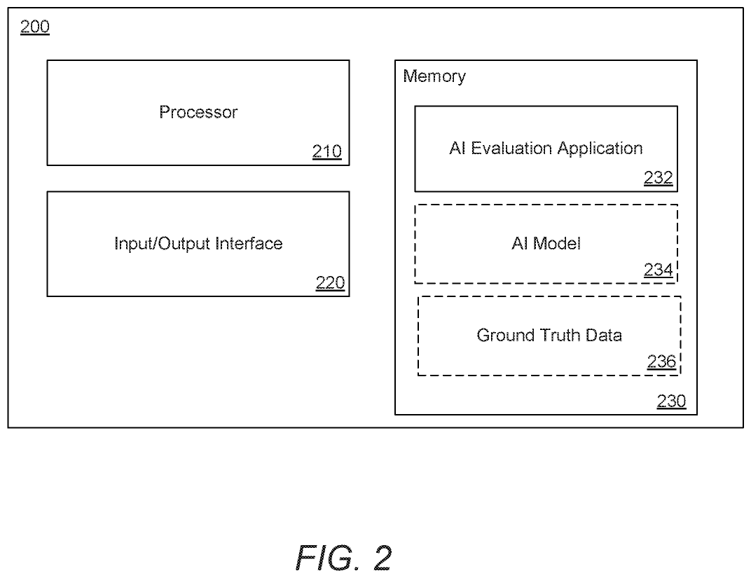

AI evaluation servers are computing devices capable of obtaining ground truth data from collection servers and using them to evaluate AI systems. In numerous embodiments, the AI evaluation server both evaluates AI systems and maintains a registry of evaluated AI systems that can indicate which system is recommended for a given application. An AI evaluation server in accordance with an embodiment of the invention is illustrated in FIG. 2.

AI evaluation server 200 includes a processor 210. Processors are any circuit capable of performing logical calculations, including, but not limited to, central processing units (CPUs), graphics processing units (GPUs), field-programmable gate arrays (FPGAs), application-specific integrated circuits (ASICs), and/or any other circuit as appropriate to the requirements of specific applications of embodiments of the invention. AI evaluation server 200 further includes an input/output (I/O) interface (220). The I/O interface is capable of sending and receiving data to external devices, including, but not limited to, collection servers. The AI evaluation server also includes a memory 230. The memory can be implemented as volatile memory, nonvolatile memory, and/or any combination thereof. The memory 230 contains an AI evaluation application 232. In numerous embodiments, the memory 230 further includes at least one AI model 234 to be tested, and ground truth data 236 received from collection servers.

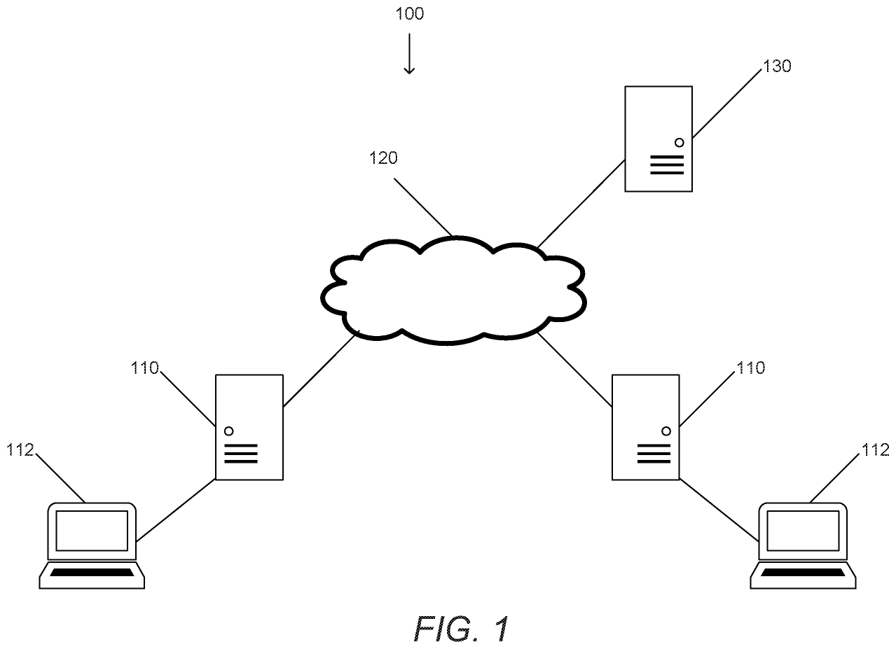

While a particular AI evaluation system and a particular AI evaluation server are illustrated in FIGS. 1 and 2, respectively, one of ordinary skill in the art can appreciate that any number of different architectures can be used as appropriate to the requirements of specific applications of embodiments of the invention without departing from the scope or spirit of the invention. Processes for evaluating AI systems are discussed in further detail below.

### AI Evaluation Processes

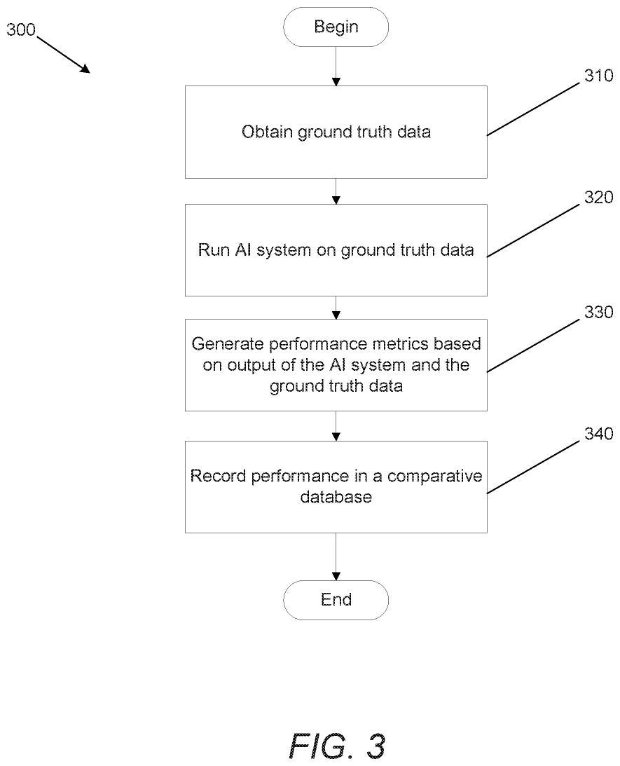

AI evaluation processes involve collecting ground truth data from many different institutions that measure similar phenomena with their individual tools and idiosyncrasies, and using that data to test the robustness and validity of AI systems in different environments and for different purposes. For example, in numerous embodiments, radiological imaging data for a particular condition can be collected at various institutions and AI image classifiers can be tested to determine their relative effectiveness. Such evaluation can be part of routine clinical workflow of all patients suitable for AI assistance, but collecting AI evaluation metrics in routine clinical workflow is often challenging for various technical reasons. For example, variation in terminology across hospitals prevents compiling the value for the same metric across different sites, and presently there is an inability to track edits that radiologists make to local AI system outputs which are shown on the images as image annotations. Collection methods described herein can address these issues. Turning now to FIG. 3, a process for evaluating AI systems in accordance with an embodiment of the invention is illustrated.

Process 300 includes obtaining (310) ground truth data from various institutions. In numerous embodiments, the ground truth data is obtained from collection servers at various institutions. In many embodiments, the ground truth data includes radiology images annotated by a radiologist. In various embodiments, the ground truth data can include outputs of an AI system utilized at the originating institution and/or the agreement/disagreement with the AI system output by a radiologist. In many embodiments, the ground truth data includes both the terminology used to describe the diagnoses or observations in the images, and image annotations that outline or point to abnormalities in the images. The former tends to vary across hospitals because it is generally conveyed as narrative text with no standardization or enforcement of standard terminology. The latter comprises edits that radiologists make to annotations produced by local AI systems so as to indicate the correct markings on the images to correctly identify the abnormalities.

In many embodiments, a computerized process to link the variety of terms that hospitals use to describe the same disease and/or imaging observation to the same term is included in the process, In numerous embodiments, a standardized ontology such as, but not limited to, RadLex is used as part of the linking process. In various embodiments, a module is used that maps uncontrolled text terms describing diseases and imaging observations that are output from AI algorithms to ontologies. This can be accomplished by generating word embeddings that are learned from a large number of the outputs of AI algorithms and corresponding ontology terms that are manually curated in a training set, and training a machine learning algorithm to generate the mappings. These mappings then, when encountering uncontrolled terms from AI outputs, can replace them with an standard ontology term, enabling unification of different ways different AI systems at different hospitals record diagnosis and imaging observations aspects of the gold standard. Further, to record corrections made to local AI system outputs, machine learning methods can be trained to transcode the annotations output from an AI system in different formats to a standardized format such as, but not limited to, the Annotation and Image (AIM) markup format.

The AI evaluation server runs (320) the AI system to be evaluated on the ground truth institutional data and generates (330) performance metrics based on the output of the AI system and the ground truth data. In numerous embodiments, the success of the AI is calculated based on predictions generated by the AI system with the reference standard for a particular case in the ground truth data. The performance of the AI system is recorded (340) in a comparative database along with the performance of other evaluated AI systems. In various embodiments, the abilities of different AI systems are tested only with cases in the ground truth data that contain conditions that the AI system is trained to classify. However, in various embodiments, other cases can be provided to the AI system to test validity and robustness.

Systems and methods described herein can be used as part of the evaluation of any AI algorithm by any clinical practice before deploying it for use in patients. In addition, systems and methods described herein can be regularly used once an AI system is deployed to regularly check that performance is meeting required goals (i.e., monitoring of performance).

Although specific methods for AI evaluation are discussed above with respect to FIG. 3, many different methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. An AI evaluation system comprising:
a plurality of collection servers;
an AI evaluation server connected to the plurality of collection servers, comprising:
at least one processor; and
a memory, containing an AI evaluation application that directs the processor to:
obtain a plurality of ground truth data from the plurality of collection servers, where the ground truth data comprises a plurality of image and annotation pairs;
generate a first plurality of outputs by providing a first AI system with images from the plurality of image and annotation pairs;
compare the first plurality of outputs with annotations from the plurality of image and annotation pairs;
generate a first ranking metric of the first AI system based on the comparison; and
store the first ranking metric in a database.

2. The AI evaluation system of claim 1, where the AI evaluation application further directs the processor to:
generate a second plurality of outputs by providing a second AI system with images from the plurality of image and annotation pairs;
compare the second plurality of outputs with annotations from the plurality of image and annotation pairs;
generate a second ranking metric of the second AI system based on the comparison;
store the second ranking metric in the database; and
recommend an AI system for a particular purpose based on the ranking metrics in the database in response to a query.

3. The AI evaluation system of claim 1, wherein images in the plurality of image and annotation pairs are radiology images.

4. The AI evaluation system of claim 1, wherein the ground truth data conforms to the Annotation and Image Markup (AIM) file standard.

5. The AI evaluation system of claim 1, wherein collection servers in the plurality of collection servers are hospital servers.

6. The AI evaluation system of claim 1, wherein the ground truth data is deidentified.

7. The AI evaluation system of claim 1, wherein an annotation of an image and annotation pair identifies whether a disease indicator is present in an image in the image and annotation pair.

8. The AI evaluation system of claim 1, wherein an annotation of an image and annotation pair is the output of the first AI system and an agree/disagree indicator by a radiologist of the output of the first AI system.

9. The AI evaluation system of claim 1, wherein the ground truth data is divided into different classifications by image type.

10. The AI evaluation system of claim 1, further comprising an input device connected to at least one collection server in the plurality of collection servers, where the input device is running the ePAD application.

11. A method of evaluating AI comprising:
obtaining a plurality of ground truth data from a plurality of collection servers, where the ground truth data comprises a plurality of image and annotation pairs, using an AI evaluation server;
generating a first plurality of outputs by providing a first AI system with images from the plurality of image and annotation pairs, using the AI evaluation server;
comparing the first plurality of outputs with annotations from the plurality of image and annotation pairs, using the AI evaluation server;
generating a first ranking metric of the first AI system based on the comparison, using the AI evaluation server; and
storing the first ranking metric in a database, using the AI evaluation server.

12. The method of evaluating AI systems of claim 11, further comprising:
generating a second plurality of outputs by providing a second AI system with images from the plurality of image and annotation pairs, using the AI evaluation server;
comparing the second plurality of outputs with annotations from the plurality of image and annotation pairs, using the AI evaluation server;
generating a second ranking metric of the second AI system based on the comparison, using the AI evaluation server;
storing the second ranking metric in the database, using the AI evaluation server; and
recommending an AI system for a particular purpose based on the ranking metrics in the database in response to a query, using the AI evaluation server.

13. The method of evaluating AI systems of claim 11, wherein images in the plurality of image and annotation pairs are radiology images.

14. The method of evaluating AI systems of claim 11, wherein the ground truth data conforms to the Annotation and Image Markup (AIM) file standard.

15. The method of evaluating AI systems of claim 11, wherein collection servers in the plurality of collection servers are hospital servers.

16. The method of evaluating AI systems of claim 11, wherein the ground truth data is deidentified.

17. The method of evaluating AI systems of claim 11, wherein an annotation of an image and annotation pair identifies whether a disease indicator is present in an image in the image and annotation pair.

18. The method of evaluating AI systems of claim 11, wherein an annotation of an image and annotation pair is the output of the first AI system and an agree/disagree indicator by a radiologist of the output of the first AI system.

19. The method of evaluating AI systems of claim 11, wherein the ground truth data is divided into different classifications by image type.

20. The method of evaluating AI systems of claim 11, further comprising receiving ground truth data using an input device connected to at least one collection server in the plurality of collection servers, where the input device is running the ePAD application.
